SEQUENCE LISTING (8 sequences)

SEQ ID NO: 1 - protein, 303 aa, Candida utilis
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 2 - protein, 304 aa, Sus scrofa
MAHYRNDYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGRITGTVKRKLTSRL

SEQ ID NO: 3 - protein, 304 aa, Papio hamadryas
MADYHNNYKKNDELEFVRTGYGKDMVKVLHIQRDGKYHSIKEVATSVQLTLSSKKDYLHGDNSDIIPTDTIKNTVHVLAKFKGIKSIEAFGVNICEYFLSSFNHVIRAQVYVEEIPWKRLEKNGVKHVHAFIHTPTGTHFCEVEQLRSGPPVITSGIKDLKVLKTTQSGFEGFIKDQFTTLPEVKDRCFATQVYCKWRYHQCRDVDFEATWGTIRDLVLEKFAGPYDKGEYSPSVQKTLYDIQVLSLSRVPEIEDMEISLPNIHYFNIDMSKMGLINKEEVLLPLDNPYGKITGTVKRKLSSRL

SEQ ID NO: 4 - protein, 304 aa, synthetic construct
  REGION 1..304: note = PKS chimera
MAHYRNDYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRL

SEQ ID NO: 5 - protein, 298 aa, synthetic construct
  REGION 1..298: note = PKS?N chimera
DYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRL

SEQ ID NO: 6 - protein, 13 aa, synthetic construct
  REGION 1..13: note = Pig-TTKS (Fragment 44-56)
ATTVQLTLSSKKD

SEQ ID NO: 7 - protein, 299 aa, synthetic construct
  REGION 1..299: note = Pig-TTKS-?N
MTYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRL

SEQ ID NO: 8 - protein, 298 aa, synthetic construct
  REGION 1..298: note = Pig-TTKS-?N without starting Met
TYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRL

(19) United States
(12) Patent Application Publication
(10) Pub. No.: US 2026/0284151 A1
(43) Pub. Date: Sep. 24, 2026

(54) COMBINATION THERAPIES OF PREDNISONE AND URICASE MOLECULES AND USES THEREOF

(71) Applicant: HORIZON THERAPEUTICS USA, INC., DEERFIELD, IL (US)

(72) Inventors: Jeffrey D. KENT, DEERFIELD, IL (US); Brian LAMOREAUX, LAKE FOREST, IL (US)

(73) Assignee: HORIZON THERAPEUTICS USA, INC., DEERFIELD, IL (US)

(21) Appl. No.: 19/389,928

(22) Filed: Nov. 14, 2025

Related U.S. Application Data
(63) Continuation of application No. 16/347,900, filed on Nov. 10, 2017, now Pat. No. 12,496,331.
(60) Provisional application No. 62/421,079, filed on Nov. 11, 2016.

(51) Int. Cl. A61K 38/44; A61K 9/00; A61K 31/381; A61K 31/519; A61K 31/573; A61P 37/00

(52) U.S. Cl. CPC A61K 38/443; A61K 9/0019; A61K 31/381; A61K 31/519; A61K 31/573; A61P 37/00; C12Y 107/03003

(57) ABSTRACT

Described herein are methods for reducing an antibody response to uricase therapy, improving the treatment of gout, reducing uric acid levels, and preventing and/or delaying infusion reactions. The methods may include administration of a uricase and a steroid as described herein.

### CROSS REFERENCE TO RELATED APPLICATIONS

This application is a continuation of U.S. patent application Ser. No. 16/347,900, filed May 7, 2019; which is the United States National Phase of International Patent Application No. PCT/US17/061126, filed Nov. 10, 2017; which claims the benefit of U.S. Provisional Application No. 62/421,079, filed Nov. 11, 2016, the disclosure of all of which are hereby incorporated by reference in their entirety.

### INCORPORATION OF SEQUENCE LISTING

The sequence listing that is contained in the file named “10774-US03-CNT_Seglisting,” which is 11,735 bytes as measured in Microsoft Windows operating system and was created on Nov. 4, 2025, is filed electronically herewith and incorporated herein by reference.

### FIELD OF THE DISCLOSURE

The disclosure generally relates to methods for reducing an antibody response to uricase therapy for treatment of gout.

### BACKGROUND OF THE DISCLOSURE

Hyperuricemia is a condition characterized by abnormally high level of uric acid in the blood. Uric acid (UA) is an end metabolite in the human purine catabolic pathway. Under physiological pH conditions, uric acid exists largely as urate, the ion form of UA. The amount of urate in the body depends on the balance between the amount of purines obtained by food intake, the amount of urate synthesized within the body, and the amount of urate that is excreted in urine or through the gastrointestinal tract. In humans, the upper end of the normal range is about 360 μmol/L (about 6 mg/dL) for women and about 400 μmol/L (about 6.8 mg/dL) for men. When the concentration of serum uric acid (SUA) is above the biochemical limit of solubility, e.g., about 6.8 mg/dL, monosodium urate crystals can precipitate in tissues. After years of persistent hyperuricemia, accumulation of monosodium urate crystals can cause symptoms of gout, such as acute inflammation of joints (gout flare), formation of gout tophi, gouty arthritis, and UA nephropathy (including UA renal stones). A total of about 8.3 million patients have been diagnosed with gout in the United States. The need exists for improving therapies that decrease the level of uric acid in the blood, which can be used to treat hyperuricemia and related conditions.

### SUMMARY OF THE DISCLOSURE

This disclosure is based, at least in part, on the discovery that a combination of a steroid and a uricase molecule improves treatment of hyperuricemia and related conditions in a subject, e.g., relative to a uricase molecule alone. In some embodiments, one or more treatment cycles, each treatment cycle comprising administration of a steroid and a uricase molecule. In some embodiments, the steroid molecule is administered prior to, concurrently with, or following the uricase molecule.

Without wishing to be bound by theory, the novel combinations disclosed herein are believed to increase the responsiveness of the subject to a uricase therapy, e.g., a therapy comprising the uricase molecule, e.g., by preventing, e.g., delaying and/or reducing, an infusion reaction. Accordingly, methods, compositions, dosage regimens, and kits are disclosed herein to improve the efficacy of a uricase therapy.

In one aspect, the disclosure provides a method of reducing uric acid levels in a subject in need thereof, comprising: administering to the subject a uricase molecule, and a steroid; wherein the uricase molecule and the steroid together reduce the uric acid levels in the subject to an extent greater than the reduction seen with administration of a uricase and/or steroid alone. In one embodiment, the steroid is administered prior to administration of the uricase molecule. In another embodiment, the steroid is administered 10 days, 9 days, 8 days, 7 days, 6 days, 5 days, 4 days, 4 days, 3 days, 2 days, 1 day, 18 hours, 12 hours, 6 hours, 4 hours, 2 hours, or 1 hour prior to administration of the uricase molecule. In another embodiment, the steroid is administered concurrently with the uricase molecule. In another embodiment, the steroid is administered after administration of the uricase molecule. In another embodiment, the steroid is administered 10 days, 9 days, 8 days, 7 days, 6 days, 5 days, 4 days, 4 days, 3 days, 2 days, 1 day, 18 hours, 12 hours, 6 hours, 4 hours, 2 hours, or 1 hour after administration of the uricase molecule.

In another aspect, the disclosure provides a method of reducing uric acid levels in a subject in need thereof, comprising: administering to the subject a uricase molecule and a steroid, wherein the uricase molecule is administered in a dose amount and/or frequency sufficient to reduce uric acid levels in the subject; discontinuing the administration of the uricase molecule after the uric acid levels are reduced in response to the uricase molecule; and administering a second non-uricase urate-lowering therapy after discontinuing the administration of the uricase molecule. In one embodiment, the second non-uricase urate-lowering therapy is an oral urate-lowering agent (ULA) selected from a xanthanine oxidase inhibitor, allopurinol, and febuxostat.

In another embodiment, the subject's serum uric acid level is ≤6 mg/dL after discontinuing the uricase molecule. In another embodiment, the subject's serum uric acid level is ≤6 mg/dL for six months or more after discontinuing the uricase molecule.

In another embodiment, the treatment period comprises administering a dose of the uricase molecule once every 4-10 days. In another embodiment, the treatment period comprises administering a dose of the uricase molecule once every 7 days. In another embodiment, the treatment period comprises: administering a first dose of the uricase molecule and a first dose of the steroid; and administering a subsequent dose of the uricase molecule and the steroid every 14 days thereafter for the duration of the treatment period. In another embodiment, the method further comprising administering an additional dose of the uricase molecule and the steroid 7 days after the first dose.

In another embodiment, the first dose of the uricase molecule is 12 mg, and the subsequent doses of the uricase molecule are 8 mg; and the first dose and the subsequent doses of the steroid are from about 40 mg to about 200 mg. In another embodiment, the first dose of the uricase molecule is 16 mg, and the subsequent doses of the uricase molecule are 8 mg; and the first dose and the subsequent doses of the steroid are from about 40 mg to about 200 mg. In another embodiment, the uricase molecule is administered at a dose of about 4 mg, about 5 mg, about 6 mg, about 7 mg, about 8 mg, about 12 mg, or about 16 mg; and the steroid is administered at a dose of from about 40 mg to about 200 mg. In another embodiment, the uricase molecule is administered at a dose of about 8 mg, and the steroid is administered at a dose of from about 40 mg to about 200 mg. In another embodiment, the first dose of the uricase molecule and the steroid is administered to patients weighing greater than 120 kg.

In another embodiment, the treatment period comprises administering 1 dose, 2 doses, 3 doses, 4 doses, or 5 doses of the uricase molecule and the steroid. In another embodiment, the treatment period comprises administering 3 doses of the uricase molecule and the steroid.

In another embodiment, the administration of the uricase and the steroid together decreases immune clearance of the uricase molecule, reduces antibody generation in response to the uricase molecule, reduces an immune response to the uricase molecule, increases immunosuppression, increase antigen-specific immunosuppression, and/or reduces an infusion reaction.

In another embodiment, the method comprises administering a dose of the uricase molecule and the steroid once every 11-18 days. In another embodiment, the method comprises administering a dose of the uricase molecule and the steroid once every 14 days.

In another embodiment, the method comprises: administering the uricase molecule at a dose of about 4 mg, about 5 mg, about 6 mg, about 7 mg, about 8 mg, or about 12 mg; and administering the steroid at a dose of from about 40 mg to about 200 mg. In another embodiment, the method comprises administering the uricase molecule at a dose of about 8 mg and the steroid at a dose of from about 40 mg to about 200 mg.

In another embodiment, the method comprises administering 4 doses, 5 doses, 6 doses, 7 doses, 8 doses, 9 doses, 10 doses, 11 doses, 12 doses, 24 doses, or 48 doses of the uricase molecule and the steroid. In another embodiment, the method comprises administering 4 doses of the uricase molecule and the steroid. In another embodiment, the method comprises administering 12 doses of the uricase molecule and the steroid.

In another embodiment, the serum uric acid levels are reduced to ≤6 mg/dL as a result of treatment with the uricase and the steroid. In another embodiment, the serum uric acid levels are reduced to ≤6 mg/dL for at least six months.

In another embodiment, the uricase molecule is a uricase conjugate. In another embodiment, the uricase conjugate comprises one or more of a polyether, polyester, polyamino acid, a polycarbonate, a polyacetal, a polyketal, a polysaccharide, or a combination thereof. In another embodiment, the polyether is selected from polyethylene glycol and polypropylene glycol.

In another embodiment, the uricase molecule comprises a fungal or microbial uricase. In another embodiment, the uricase is selected from Aspergillus flavus, Arthrobacter globfornis, Bacillus, sp., and Candida utilis uricase. In another embodiment, the uricase molecule comprises an invertebrate uricase. In another embodiment, the uricase is selected from the group consisting of Drosophila melanogaster and Drosophila pseudoobscura uricase. In another embodiment, the uricase molecule comprises a plant uricase. In another embodiment, the uricase is from Glycine max. In another embodiment, the uricase molecule comprises a mammalian uricase. In another embodiment, the mammalian uricase is selected from the group consisting of a bovine, a porcine, an ovine, and a baboon uricase. In another embodiment, the uricase molecule comprises a chimeric uricase comprising amino acids from two or more mammalian uricases. In another embodiment, the uricase molecule comprises a recombinant pig-baboon chimeric uricase.

In another embodiment, the uricase molecule comprises a sequence that is at least 90% identical to SEQ ID NO:8. In another embodiment, the uricase molecule comprises the amino acid sequence of SEQ ID NO:8. In another embodiment, the uricase molecule comprises a uricase that is truncated by 3-10 amino acids at its amino terminus, its carboxy terminus, or both. In another embodiment, the uricase molecule comprises a uricase truncated by 4-13 amino acids at its amino terminus, its carboxy terminus, or both. In another embodiment, the uricase molecule comprises a uricase truncated by 6 amino acids at its amino terminus, its carboxy terminus, or both. In another embodiment, the uricase molecule comprises a pig uricase having the amino acid sequence of SEQ ID NO:2. In another embodiment, the uricase molecule comprises a pig uricase having the amino acid sequence of SEQ ID NO:2 modified by amino acid substitutions D7T, S46T, R291K, and T301S. In another embodiment, the uricase molecule comprises the amino acid sequence of any one of SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

In another embodiment, the uricase molecule is pegylated. In another embodiment, the uricase molecule comprises four identical non-covalently-bound uricase monomeric subunits. In another embodiment, the uricase molecule is a tetramer.

In another embodiment, the uricase molecule comprises the amino acid sequence of des-(1-6)-[7-threonine, 46-threonine, 291-lysine, 301-serine]pig-baboonuricase. In another embodiment, the uricase molecule comprises monomethoxypoly (ethylene glycol) modified recombinant mammalian uricase. In another embodiment, the uricase molecule is pegloticase (KRYSTEXXA®).

In another embodiment, the subject is hyperuricemic. In another embodiment, the subject has gout, chronic gout, refractory gout, or tophaceous gout. In another embodiment, the subject was previously treated with one or more of an oral urate-lowering therapy, a xanthanine oxidase inhibitor, allopurinol, and febuxostat. In another embodiment, the subject has a body weight ≥about 80 kg, 90 kg, 100 kg, 110 kg, or 120 kg. In another embodiment, the subject is ≥55, 60, 65, or 70 years old.

In another embodiment, the method includes ameliorating or reducing at least one symptom or measurable physiological parameter of hyperuricemia in the subject. In another embodiment, the method includes ameliorating or reducing at least one symptom or measurable physiological parameter of gout in the subject. In another embodiment, the method includes reducing the subject's uric acid levels, reducing incidence of gout flares, reducing inflammation of the joints, reducing formation of gout tophi, reducing gout arthritis, and/or reducing uric acid nephropathy. In another embodiment, the method includes reducing the subject's C-reactive protein levels. In another embodiment, the method includes reducing the subject's Health Assessment Questionnaire Disability Index (HAQ-DI). In another embodiment, the method includes ameliorating or reducing gout, chronic gout, refractory gout, or tophaceous gout. In another embodiment, the method includes delaying or preventing an infusion reaction in the subject. In another embodiment, the method includes reducing or preventing an antibody response to the uricase in the subject.

In another embodiment, the method further comprises administering at least one immunosuppressant prior to or concurrently with the administration of the uricase molecule. In another embodiment, the method further comprises administering at least one anti-inflammatory compound prior to or currently with the administration of the uricase molecule. In another embodiment, the inflammatory compound is selected from a corticosteroid, an antihistamine, colchicine, and a nonsteroidal anti-inflammatory drug (NSAID). In another embodiment, the corticosteroid is selected from the group consisting of predisone, hydrocortisone, methylprednisolone, Depo-Medrol, Solu-Medrol.

In another embodiment, the uricase molecule and the steroid are administered in a pharmaceutical composition. In another embodiment, the uricase molecule and the steroid are administered in a nanocarrier formulation. In another embodiment, the uricase molecule and the steroid are administered by intravenous infusion.

In another aspect, the disclosure provides a kit comprising: a uricase molecule; a steroid; and instructions for use in the methods described herein.

In another aspect, the disclosure provides a method of treating chronic gout in a subject, comprising: administering to the subject a uricase molecule and a steroid; wherein the uricase and the steroid together reduce uric acid levels in the subject.

In another aspect, the disclosure provides a method of treating chronic gout in a subject, comprising: administering to the subject a uricase molecule and a steroid, wherein the uricase molecule is administered in a dose amount and/or frequency sufficient to reduce uric acid levels in the subject; discontinuing the administration of the uricase molecule after the uric acid levels are reduced in response to the uricase molecule; and administering a second non-uricase urate-lowering therapy after discontinuing the administration of the uricase molecule.

Additional features and embodiments of the methods, compositions, dosage regimens, and kits disclosed herein include one or more of the following.

### Combination Therapies/Administration

In some embodiments, a steroid is administered prior to, concurrently with, or following, a uricase conjugate. In some embodiments, the uricase conjugate may be administered at a dose of about 4 mg, about 5 mg, about 6 mg, about 7 mg, about 8 mg, or about 12 mg, e.g., about 8 mg.

In some embodiments, a steroid as described herein may be predisone, hydrocortisone, methylprednisolone, Depo-Medrol, Solu-Medrol. One of skill will recognize that any steroid may be useful in accordance with the disclosure as described herein, in order to achieve the desired results. For example, a prednisone molecule, e.g., the methylprednisolone, may be administered in an amount ranging from about 0.1 mg/kg to about 5 mg/kg, about 0.2 mg/kg to about 4 mg/kg, about 0.3 mg/kg to about 3 mg/kg, about 0.4 mg/kg to about 2 mg/kg, or about 0.5 mg/kg to about 1.5 mg/kg, or typically about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, or about 1.5 mg/kg. In other embodiments, the methylprednisolone is administered as a flat dose, e.g., about 1 mg to about 500 mg, about 10 mg to about 400 mg, about 20 mg to about 300 mg, about 30 mg to about 200 mg, or about 50 mg to about 150 mg, or typically about 50 mg, about 60 mg, about 70 mg, about 80 mg, about 90 mg, about 100 mg, about 110 mg, about 120 mg, about 130 mg, about 140 mg, or about 150 mg.

In some embodiments, a steroid as described herein may be a hydrocortisone, a methylprednisolone, a prednisone, or any other steroid deemed appropriate in accordance with the present disclosure. In some embodiments, a methylprednisolone as described herein may be, for example, Depo-Medrol, Solu-Medrol, or the like. Dosages for a steroid as described herein may vary as appropriate. For example, hydrocortisone may be administered intravenously (IV) at a dose of 200 mg every two weeks immediately before pegloticase infusions. In some embodiments, methylprednisolone may be administered IV at a dose of 40 mg every two weeks immediately before pegloticase infusions. In some embodiments, methylprednisolone may be administered IV at a dose of 80 mg every two weeks immediately before pegloticase infusions. While such dosages may represent common dosages, one of skill in the art will recognize that other dosages may be give as appropriate and as deemed beneficial in accordance with the present disclosure.

In some embodiments, prednisone may be administered with pegloticase as a prophylactic treatment for gout flare in order to prevent gout flare. In such cases, prednisone may be administered orally (PO) at a dosage of 5 or 10 mg daily. As would be understood by one of skill in the art, in some embodiments, any route of administration may be used as appropriate for the particular drug treatment and/or dosage.

In some embodiments, the prednisone molecule, e.g., the methylprednisolone, is administered prior to the uricase molecule. In some embodiments, the prednisone molecule is administered about 10 minutes or more, about 20 minutes or more, about 30 minutes or more, about 40 minutes or more, about 50 minutes or more, about 1 hour or more, about 2 hours or more, or about 3 hours or more before administration of the uricase molecule. In other embodiments, the prednisone molecule is administered about 10 minutes, about 20 minutes, about 30 minutes, about 40 minutes, about 50 minutes, about 1 hour, about 2 hours, or about 3 hours before administration of the uricase molecule. In other embodiments, the administration of the prednisone molecule and the uricase molecule partially overlap.

In one embodiment, the prednisone molecule is administered intravenously (IV), intramuscularly (IM), by infusion (IV infusion), orally, rectally, or topically.

In some embodiments, the treatment cycle is repeated once every 11 days, 12 days, 13 days, 14 days, 15 days, 16 days, 17 days, or 18 days, e.g., every 14 days, e.g., every other week. In one embodiment, dosage regimen is repeated once every other week.

In some embodiments, the dosage regimen comprises one or more first treatment cycles of prednisone molecule and a uricase molecule, and one or more second treatment cycles of a uricase molecule without prednisone.

In some embodiments, dosage regimen includes 7, 10, 12, 24, 48, or more treatment cycles.

### Uricase Molecules

In some embodiments, the uricase molecule is a naturally occurring uricase, or a variant of a naturally occurring uricase (e.g., a mutated form, a truncated form, fragment form, a chimeric form, or a derivative form).

In some embodiments, the uricase molecule is recombinant.

In some embodiments, the uricase molecule is a tetramer, e.g., a homotetramer.

In some embodiments, the uricase molecule is a mammalian uricase. In some embodiments, the uricase molecule is porcine, bovine, or ovine, or baboon uricase, or a chimeric combination thereof. In some embodiments, the uricase molecule comprises substantially the same sequence of porcine, bovine, ovine, or baboon uricase, or a chimeric combination thereof.

In some embodiments, the uricase molecule is a fungal or a microbial uricase, e.g., a yeast uricase. In some embodiments, the fungal or microbial uricase is from Aspergillus flavus, Arthrobacter globiformus, or Candida utilis, or comprises substantially the same sequence as any of said fungal or microbial uricases.

In some embodiments, the uricase molecule is a plant uricase.

In some embodiments, the uricase molecule is a chimera. In some embodiments, the uricase molecule comprises a portion of porcine liver and baboon liver uricase.

In some embodiments, the uricase molecule comprises a mammalian uricase amino acid sequence truncated at the amino terminus or the carboxy terminus, or both, (e.g., by about 1-13 amino acids), said truncation being relative to a naturally occurring pig uricase having an amino acid sequence of SEQ ID NO:2. In some embodiments, the uricase molecule comprises an amino acid substitution with threonine at position 7 (D7T), an amino acid substitution with threonine at position 46 (S46T), an amino acid substitution with lysine at position 291 (R291K) and an amino acid substitution with serine at position 301 (T301S), said amino acid substitution being relative to a naturally occurring pig uricase having an amino acid sequence of SEQ ID NO:2.

In some embodiments, the amino terminal amino acid of the uricase molecule is alanine, glycine, proline, serine, or threonine. In some embodiments, the amino terminal amino acid of the uricase molecule is threonine.

In some embodiments, the uricase molecule comprises the amino acid sequence of des-(1-6)-[7-threonine, 46-threonine, 291-lysine, 301-serine]pig-baboon uricase.

In some embodiments, the uricase molecule comprises the amino acid sequence of SEQ ID NO:8, or an amino acid sequence substantially identical thereto (e.g., 95-98% identical).

In some embodiments, the uricase molecule comprises four identical non-covalently-bound uricase monomeric subunits.

In some embodiments, the uricase molecule is about 130-140 kDa, e.g., about 135-137 kDa (e.g., about 136.8 kDa).

In some embodiments, the uricase molecule further comprises a polymer. In some embodiments, the uricase molecule is in the form of a conjugate, e.g., a conjugate that includes a uricase sequence (e.g., a mutated form, a truncated form, fragment form, a chimeric form, of a uricase) coupled to, e.g., directly or indirectly (e.g., via a linker) to a polymer.

In some embodiments, the polymer comprises one or more of a polyether, polyester, polyamino acid, a polycarbonate, a polyacetal, a polyketal, a polysaccharide, or a combination thereof. In some embodiments, the polyether comprises polyethylene glycol or polypropylene glycol.

In some embodiments, the conjugate comprises 1 to 40 polymer molecules, e.g., polyethylene glycol molecules, per monomer of the uricase molecule. In other embodiments, the conjugate comprises 5 to 20 polymer molecules, e.g., polyethylene glycol molecules, per monomer of the uricase molecule. In further embodiments, the conjugate comprises 5 to 12 polymer molecules, e.g., polyethylene glycol molecules, per monomer of the uricase molecule. In some embodiments, the conjugate comprises about 10 polymer molecules, e.g., polyethylene glycol molecules, per monomer of the uricase molecule.

In some embodiments, each polymer molecule, e.g., polyethylene glycol molecule, has a molecular weight between about 1 kDa and 100 kDa. In other embodiments, each polymer molecule, e.g., polyethylene glycol molecule, has a molecular weight between about 5 kDa and 50 kDa. In further embodiments, each polymer molecule, e.g., polyethylene glycol molecule, has a molecular weight between about 5 kDa and 20 kDa. In some embodiments, each polymer molecule, e.g., polyethylene glycol molecule, has a molecular weight of about 10 kDa.

In some embodiments, the conjugate has a polymer weight of about 500-600 kDa, e.g., about 540-560 kDa.

In some embodiments, the conjugate the uricase molecule is derivatized using monomethoxypoly(ethylene glycol)-p-nitrophenyl carbonate (mPEG-NPC).

In some embodiments, the conjugate of the uricase molecule comprises a methoxy PEG (mPEG). In one embodiment, each uricase sequence includes a monomethoxypoly (ethylene glycol) (mPEG), e.g., a 5-50 kDa, e.g., a 10 kDa, mPEG moiety.

In some embodiments, the uricase molecule, e.g., the uricase conjugate, is in a pharmaceutical composition, e.g., formulation with a pharmaceutically acceptable carrier or excipient.

In some embodiments, the uricase molecule, e.g., the uricase conjugate, is in a nanocarrier formulation. In further embodiments, the nanocarrier formulation comprises a lipid nanoparticle, a liposome, a polymeric nanoparticle, a dendrimer, or a surfactant based emulsion. In additional embodiments, the polymeric nanoparticle comprises one or more of a polyether, polyester, polyamino acid, a polycarbonate, a polyacetal, a polyketal, a polysaccharide, or a combination thereof. In some embodiments, the polyether comprises polyethylene glycol or polypropylene glycol.

In further embodiments, the pharmaceutical composition, e.g., formulation, includes an immunosuppressant. In additional embodiments, the immunosuppressant is an mTOR inhibitor, e.g., rapamycin or a rapamycin analogue.

In some embodiments, the uricase molecule, e.g., the uricase conjugate, is pegloticase (KRYSTEXXA®).

### Prednisone Molecule

In some embodiments, the prednisone molecule is prednisone, or a variant, metabolite or derivative thereof (including a pharmaceutically acceptable form thereof, e.g., a salt thereof).

In one embodiment, the prednisone molecule is compound 17,21-dihydroxypregna-1,4-diene-3,11,20-trione (IUPAC name), or a variant, metabolite or derivative thereof.

In one embodiment, the prednisone molecule is a methylated derivative of prednisone, e.g., meprednisone. In one embodiment, meprednisone is compound (160)-17,21-dihydroxy-16-methylpregna-1,4-diene-3,11,20-trione (IUPAC name), or a variant, metabolite or derivative thereof (including a pharmaceutically acceptable form thereof, e.g., a salt thereof).

In one embodiment, the prednisone molecule is an active metabolite of prednisone. In one embodiment, the active metabolite of prednisone is prednisolone.

In some embodiments, the prednisone molecule is a prednisolone or a variant, metabolite or derivative thereof (including a pharmaceutically acceptable form thereof, e.g., a salt thereof), collectively referred to herein as a prednisolone molecule. In one embodiment, the prednisolone molecule is chosen from prednisolone acetate, methylprednisolone acetate, or methylprednisolone sodium succinate.

In one embodiment, the prednisolone molecule is compound (11p)-11,17,21-Trihydroxypregna-1,4-diene-3,20-dione (IUPAC name), or a variant, metabolite, or derivative thereof (including a pharmaceutically acceptable form thereof, e.g., a salt thereof).

In another embodiment, the prednisolone molecule is a variant of prednisolone, methylated at carbon 6 of the B ring.

In yet another embodiment, the prednisolone molecule is methylprednisolone, e.g., is compound (1S,2R,8S,10S,11S,14R,15S,17S)-14,17-dihydroxy-14-(2-hydroxyacetyl)-2,8,15-trimethyltetracyclo[8.7.0.0^{2,7}.0^{11,15}]heptadeca-3,6-dien-5-one (IUPAC name), or a variant or derivative thereof (including a pharmaceutically acceptable form thereof, e.g., a salt thereof).

In some embodiments, the prednisone molecule is meprednisone, prednisolone, methylprednisolone, prednisolone acetate, methylprednisolone acetate, or methylprednisolone sodium succinate.

In one embodiment, the prednisone molecule or the prednisolone molecule is not hydrocortisone.

### Additional Combination Therapies

In some embodiments, the combinations, e.g., treatment cycle, further include administering a colchicine or a non-steroidal anti-inflammatory drug. In some embodiments, the administration of the colchicine or the non-steroidal anti-inflammatory drug is started prior to administration of the uricase molecule, e.g., the uricase conjugate, e.g., 4 days, 5 days, 6 days, 7 days, 8 days, 9 days, or 10 days prior to administration of the uricase molecule, e.g., the uricase conjugate, e.g., 7 days prior to administration of the uricase molecule, e.g., the uricase conjugate. In some embodiments, colchicine is administered once a day, twice a day, or every other day, e.g., once a day. In further embodiments, colchicine is administered at a dose of 0.6 to 1.2 mg/day.

In some embodiments, the treatment cycle further comprises administering at least one additional anti-inflammatory compound prior to administration of the uricase molecule, e.g., the uricase conjugate. In further embodiments, the anti-inflammatory compound is selected from an antihistamine and a Nonsteroidal Anti-Inflammatory Drug (NSAID). In some embodiments, at least one corticosteroid, at least one antihistamine and at least one NSAID are administered. In some embodiments, the corticosteroid may include, but is not limited to, be predisone, hydrocortisone, methylprednisolone, Depo-Medrol, or Solu-Medrol.

In some embodiments, an NSAID is chosen from ibuprofen, indomethacin, naproxen, aspirin, acetaminophen, celecoxib or valdecoxib. In certain embodiments, the NSAID is acetaminophen. In some embodiments, acetaminophen is administered at a dosage of 500 to 1500 mg. In some embodiments, the acetaminophen is administered 1 to 4 hours prior to administration of the uricase molecule, e.g., the uricase conjugate.

In some embodiments, the antihistamine is selected from azatadine, brompheniramine, cetirizine, chlorpheniramine, clemastine, cyproheptadine, desloratadine, dexchlorpheniramine, dimenhydrinate, diphenhydramine, doxylamine, fexofenadine, hydroxyzine, loratadine and phenindamine. In certain embodiments, the antihistamine is fexofenadine. In some embodiments, fexofenadine is administered at a dosage of 30 to 180 mg. In certain embodiments, the fexofenadine is administered 1 to 4 hours prior to administration of the uricase molecule, e.g., the uricase conjugate. In some embodiments, the fexofenadine is administered the night before administration of the uricase molecule, e.g., the uricase conjugate.

### Pharmaceutical Compositions

In some embodiments, the uricase molecule, e.g., the uricase conjugate, is administered by injection, e.g., subcutaneously or intravenously. In one embodiment, the uricase molecule, e.g., the conjugate, is administered by subcutaneous injection.

In some embodiments, the uricase molecule, e.g., the uricase conjugate, is provided in a pharmaceutical composition comprising about 4 to about 12, e.g., about 8, milligrams of conjugate per milliliter of solution. In some embodiments, uricase molecule, e.g., the uricase conjugate, e.g., 8 milligrams of conjugate, is dissolved into 250 milliliters of saline solution for infusion. In further embodiments, wherein the saline solution includes 0.45% or 0.9% sodium chloride.

In some embodiments, the uricase molecule, e.g., the uricase conjugate, is administered over a 100-200, e.g., 120, minute period.

### Subjects

In some embodiments, the subject has or is likely to have hyperuricemia, e.g., gout. In some embodiments, the subject has chronic gout. In other embodiments, the subject has refractory or tophaceous gout. In further embodiments, the subject has, or has been identified as having, gout refractory to oral ULA therapy, e.g., has failed to normalize serum uric acid, and/or whose signs and symptoms are inadequately controlled with oral ULA therapy, e.g., xanthanine oxidase inhibitors.

In some embodiments, the subject is hyperuricemic, e.g., has a serum uric acid level≥6 mg/dl.

In some embodiments, subject was previously treated with oral ULA, e.g., xanthanine oxidase inhibitors, allopurinol, or febuxostat.

In some embodiments, the subject treated shows hyperuricemia, e.g., the subject has gout. In certain embodiments, the subject is refractory, or has reduced responsiveness, to a urate lowering therapy, e.g., oral ULA. In one embodiment, the subject has, or is identified as having, a lower efficacy to a uricase therapy (e.g., shows increased incidence of anti-uricase antibodies (e.g., anti-pegloticase antibodies) and/or increased incidence of an infusion reaction in response to the uricase therapy).

### Monitoring of Subjects

In some embodiments, the methods further include measuring the level of an antibody against the uricase molecule, e.g., the uricase conjugate, in the subject, e.g., a blood sample obtained from the subject. In some embodiments, the level of the antibody against the uricase molecule, e.g., the uricase conjugate, is measured by an immunosorbant assay, e.g., enzyme-linked immunosorbant assay (ELISA). In some embodiments, the level of the antibody against the uricase molecule, e.g., the uricase conjugate, is measured before administration of the conjugate, e.g., 72, 48, 24, 12, 6, 4, or 2 hours before administration of the uricase molecule, e.g., the uricase conjugate. In other embodiments, the level of the antibody against the uricase molecule, e.g., the uricase conjugate, is measured after administration of the uricase molecule, e.g., the uricase conjugate, e.g., 0.5, 1, 2, 3, 4, or 5 hours after administration of the uricase molecule, e.g., the uricase conjugate.

In some embodiments, the methods further include measuring the level of uric acid, e.g., a plasma or a serum uric acid level, in the subject. In some embodiments, the level of uric acid is measured before administration of the uricase molecule, e.g., the uricase conjugate, e.g., 72, 48, 24, 12, 6, 4, or 2 hours before administration of the uricase molecule, e.g., the uricase conjugate, e.g., a trough level of uric acid. In other embodiments, the level of uric acid is measured after administration of the uricase molecule, e.g., the uricase conjugate, e.g., 0.5, 1, 2, 3, 4, or 5 hours after administration of the uricase molecule, e.g., the uricase conjugate, e.g., a peak level of uric acid.

In some embodiments, the uric acid level, e.g., the serum or plasma uric acid level, is reduced in the subject relative to a reference, e.g., relative to an untreated subject, or the subject prior to treatment or after a previous treatment. In further embodiments, the uric acid level, e.g., the serum or plasma uric acid levels, is lowered to 6.8 mg/dl or less.

In some embodiments, the subject is a subject with gout refractory to oral ULA therapy, wherein administration of the uricase molecule, e.g., the uricase conjugate, is discontinued and oral ULA therapy is administered after discontinuation of the uricase molecule, e.g., the uricase conjugate, administration, and wherein the uricase molecule, e.g., the uricase conjugate, administration is for a time and in an amount such that the uric acid level of the subject remains below 6.8 mg/dl after discontinuation. In further embodiments, the uricase molecule, e.g., the uricase conjugate, administration is for a time and in an amount such that the uric acid level of the subject remains below 6.0 mg/dl. In some embodiments, the uricase molecule, e.g., the uricase conjugate, administration is for a time and in an amount such that the uric acid level of the subject remains below 6.8 mg/dl for 6 months or more after discontinuation.

In some embodiments, the uric acid level, e.g., the serum or plasma uric acid levels, is lowered to 6.0 mg/dl or less. In some embodiments, the uric acid level is measured before dose of the conjugate and the uric acid level is lowered to 6 mg/dl or less for 3 or more consecutive doses. In further embodiments, the uric acid level, e.g., the serum or the plasma uric acid level, is lowered to 5.0 mg/dl or less.

Headings or numbered or lettered elements, e.g., (a), (b), (i) etc, are presented merely for ease of reading. The use of headings or numbered or lettered elements in this document does not require the steps or elements be performed in alphabetical order or that the steps or elements are necessarily discrete from one another.

All publications, patent applications, patents, and other references mentioned herein are incorporated by reference in their entirety.

Other features, objects, and advantages of the disclosure will be apparent from the description and drawings, and from the claims.

### BRIEF DESCRIPTION OF THE SEQUENCES

SEQ ID NO:1—Sequence of uricase from Candida utilis.

SEQ ID NO:2—Sequence of uricase from porcine.

SEQ ID NO:3—Sequence of uricase from baboon.

SEQ ID NO:4—Sequence of chimeric pig-K-S or PKS uricase.

SEQ ID NO:5—Sequence of Pig-KS-AN, which is the PKS uricase (SEQ ID NO.4) truncated by 6 amino acids at its amino terminus.

SEQ ID NO:6—Sequence of positions 44 to 56 of Pig-KS-AN (SEQ ID NO:5) with a threonine at position 46.

SEQ ID NO:7—Sequence of Pig-TTKS-AN, which is positions 8 to 287 of the amino acid sequence ofPig-KS-AN (SEQ ID NO:5).

SEQ ID NO:8—Sequence of Pig-TTKS-AN, which is positions 8 to 287 of Pig-KS-AN with the N-terminal methionine removed.

### BRIEF DESCRIPTION OF THE DRAWINGS

FIG. 1—Shows the number of infusions by corticosteroid prophylaxis type.

FIG. 2—Shows percentages of patients discontinuing therapy.

FIG. 3—Shows the mean number of infusions by prophylaxis and therapy termination.

### DETAILED DESCRIPTION OF THE DISCLOSURE

Certain terms are defined.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The term “include” or “comprise” is used interchangeably herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the term “uricase molecule” includes full-length, naturally-occurring uricase molecules, as well as variants, e.g., functional variants (e.g., truncations, fragments, mutated forms (e.g., substantially similar sequences) or derivatized form thereof), so long as at least one function and/or activity of the unmodified (e.g., naturally-occurring) molecule remains. In some embodiments, the uricase molecule is derivatized, e.g., is in the form of a conjugate, e.g., a uricase conjugate as described herein.

In some embodiments, the uricase molecule is fungal or microbial uricase, e.g., has a sequence from Aspergillus flavus, Arthrobacter globiformis, Bacillus sp. or Candida utilis, or substantially identical thereto. In some embodiments, the uricase molecule is an invertebrate uricase, e.g., from Drosophila melanogaster or Drosophila pseudoobscura. In some embodiments, the uricase molecule is a plant uricase, e.g., from Glycine max.

In an embodiment, the uricase molecule is a mammalian uricase molecule. In an embodiment, the mammalian uricase molecule comprises at least a portion of a sequence of porcine, bovine, ovine or baboon liver uricase, or a combination thereof. In an embodiment, the uricase molecule is a chimeric uricase of two or more mammalian uricases selected from a portion of two or more porcine, bovine, ovine or baboon liver uricase.

In an embodiment, the uricase molecule is truncated at the N- or C-terminus. In one embodiment, the uricase molecule is truncated by 3 to 10 amino acids, e.g., 6 amino acids, at its amino terminus. In another embodiment, the uricase molecule is truncated by 3 to 10 amino acids, e.g., 6 amino acids, at its carboxy terminus. In a further embodiment, the uricase molecule is truncated by 3 to 10 amino acids, e.g., 6 amino acids, at both its carboxy and amino termini.

In an embodiment, the amino acid mutations comprise threonine, threonine, lysine, and serine, at positions 7, 46, 291, and 301, of e.g., SEQ ID NO:8, respectively.

In an embodiment, the uricase molecule includes the amino acid sequence of SEQ ID NO:8, or a sequence 90%, 91%, 92%, 93%, 94%, 95%, 96%, 97%, 98%, 99%, or 100% identical, e.g., 95-98% identical thereto.

“Derived from” as that term is used herein, indicates a relationship between a first and a second molecule. It generally refers to structural similarity between the first molecule and a second molecule and does not connotate or include a process or source limitation on a first molecule that is derived from a second molecule.

The terms “polypeptide”, “peptide” and “protein” (if single chain) are used interchangeably herein to refer to polymers of amino acids of any length. The polymer can be linear or branched, it can comprise modified amino acids, and it can be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof, amino acid analogs having variant side chains; and all stereoisomers of any of any of the foregoing. As used herein the term “amino acid” includes both the D- or L-optical isomers and peptidomimetics.

The methods and dosages of the present disclosure encompass polypeptides and nucleic acids having the sequences specified, or sequences substantially identical or similar thereto, e.g., sequences at least 85%, 90%, 95% identical or higher to the sequence specified.

In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. One set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecules of the disclosure. BLAST protein searches can be performed with the XBLAST program, score=50, wordlength=3 to obtain amino acid sequences homologous to protein molecules of the disclosure. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

It is understood that the uricase molecules can have additional conservative or non-essential amino acid substitutions, which do not have a substantial effect on their functions.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

A “conservative mutation”, as used herein, is a mutation of one or more amino acids, at or around a position that does not substantially alter the protein's behavior. In one embodiment, the uricase molecule comprising at least one conservative mutation has the same uricase activity as does uricase molecule without such mutation. In alternate embodiments, the uricase molecule comprising at least one conservative mutation has substantially the same uricase activity, within 5% of the activity, within 10% of the activity, or within 30% of the activity of uricase molecule without such mutation.

Conservative amino acid substitution is defined as a change in the amino acid composition by way of changing amino acids of a peptide, polypeptide or protein, or fragment thereof. In some embodiments, the uricase molecule has one, two, three or four conservative mutations. The substitution is of amino acids with generally similar properties (e.g., acidic, basic, aromatic, size, positively or negatively charged, polar, non-polar) such that the substitutions do not substantially alter peptide, polypeptide or protein characteristics (e.g., charge, IEF, affinity, avidity, conformation, solubility) or activity. Typical substitutions that can be performed for such conservative amino acid substitution can be among the groups of amino acids as follows:
glycine (G), alanine (A), valine (V), leucine (L) and isoleucine (I);
aspartic acid (D) and glutamic acid (E);
alanine (A), serine (S) and threonine (T);
histidine (H), lysine (K) and arginine (R);
asparagine (N) and glutamine (Q);
phenylalanine (F), tyrosine (Y) and tryptophan (W).

The protein having one or more conservative substitutions retains its structural stability and can catalyze a reaction even though its DNA sequence is not the same as that of the original protein.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

Various aspects of the disclosure are described in further detail below. Additional definitions are set out throughout the specification.

### Uricase Molecules

The methods and dosage regimens disclosed herein include the use of uricase molecules, e.g., uricase conjugates, administered in tolerizing and therapeutic dosage regimens, alone or in combination with other therapies, to, for example, reduce uric acid levels, treat gout, prevent (e.g., delaying) infusion reactions, and/or reduce an antibody response against a uricase. Uricases suitable for the methods and dosage regimens disclosed herein include naturally occurring urate oxidases isolated from bacteria, fungi and the tissues of plants and animals, both vertebrates and invertebrates, as well as recombinant forms of uricase, including mutated, hybrid, and/or truncated enzymatically active variants of uricase.

The uricase molecule can be fungal or microbial uricase. In one aspect of this embodiment, the fungal or microbial uricase molecule can be a naturally occurring or recombinant form of uricase from Aspergillus flavus, Arthrobacter globiformis, Bacillus sp. In some embodiments, the uricase molecule is from Candida utilis (SEQ ID NO:1). Alternatively, the uricase molecule can be an invertebrate uricase, such as, for example, a naturally occurring or recombinant form of uricase from Drosophila melanogaster or Drosophila pseudoobscura. The uricase molecule disclosed herein can also be a plant uricase, for example, a naturally occurring or recombinant form of uricase from soybean root nodule (Glycine max).

Uricase molecule, as used herein, includes individual subunits, as well as the tetramer, unless otherwise indicated. In some embodiments, the uricase molecule is a homotetramer. In some embodiments, the uricase molecule comprises four identical non-covalently-bound uricase monomeric subunits.

“Truncated uricase molecule”, as used herein, refers to uricase molecules having shortened primary amino acid sequences. Amongst the possible truncations are truncations at or around the amino and/or carboxy termini. Specific truncations of this type can be such that the ultimate amino acids (those of the amino and/or carboxy terminus) of the naturally occurring protein are present in the truncated protein. Amino terminal truncations can begin at position 1, 2, 3, 4, 5 or 6. In some embodiments, the amino terminal truncations begin at position 2, thereby leaving the amino terminal methionine. This methionine can be removed by post-translational modification. In some embodiments, the amino terminal methionine is removed after the uricase is produced. In one embodiment, the methionine is removed by endogenous bacterial aminopeptidase.

A truncated uricase molecule, with respect to the full length sequence, has one or more amino acid sequences excluded. A protein comprising a truncated uricase can include any amino acid sequence in addition to the truncated uricase sequence. In some embodiments, a uricase protein comprising a truncated uricase wherein the truncation begins at position 6 (i.e., the truncated uricase begins at position 7) does not have, immediately upstream from the truncated uricase, whatever amino acid that the wild type uricase has at position 6.

Unless otherwise indicated by specific reference to another sequence or a particular SEQ ID NO, reference to the numbered positions of the amino acids of the uricase molecules described herein is made with respect to the numbering of the amino acids of the pig uricase sequence. As used herein, reference to amino acids or nucleic acids “from position X to position Y” means the contiguous sequence beginning at position X and ending at position Y, including the amino acids or nucleic acids at both positions X and Y.

In an embodiment, the uricase molecule is truncated by 4-13 amino acids at its amino terminus. In another embodiment, the uricase molecule is truncated by 4-13 amino acids at its carboxy terminus. In a further embodiment, the uricase molecule is truncated by 4-13 amino acids at both its carboxy and amino termini.

In an embodiment, the uricase molecule is truncated by 6 amino acids at its amino terminus. In another embodiment, the uricase molecule is truncated by 6 amino acids at its carboxy terminus. In a further embodiment, the uricase molecule is truncated by 6 amino acids at both its carboxy and amino termini.

One exemplary mammalian uricase molecule is recombinant pig-baboon chimeric uricase, composed of portions of the sequences of pig liver and baboon liver uricase, both of which were first determined by Wu, et al., (1989). One example of such a chimeric uricase molecule contains the first 288 amino acids from the porcine sequence (SEQ ID NO:2) and the last 16 amino acids from the baboon sequence (SEQ ID NO:3). Since the latter sequence differs from the porcine sequence at only two positions, having a lysine (K) in place of arginine at residue 291 and a serine (S) in place of threonine at residue 301, this mutant is referred to as pig-K-S or PKS uricase (SEQ ID NO:4). In some embodiments, PKS uricase is truncated by 6 amino acids at its amino terminus and is referred to as “Pig-KS-AN” (SEQ ID NO:5).

In a one embodiment, the uricase molecule comprises the amino acid sequence from position 13 to position 292 of the amino acid sequence of pig uricase (SEQ ID NO:5). In another embodiment, the uricase molecule comprises the amino acid sequence from position 8 to position 287 of the amino acid sequence of Pig-KS-AN (SEQ ID NO:5).

In another embodiment, the uricase molecule comprises the amino acid sequence from position 44 to position 56 of Pig-KS-AN comprising a threonine at position 46 (SEQ ID NO:6). This region of uricase has homology to sequences within the tunneling fold (T-fold) domain of uricase, and has within it a mutation at position 46 with respect to the native pig uricase sequence.

In a one embodiment, the uricase molecule comprising threonine, threonine, lysine, and serine, at positions 7, 46, 291, and 301 comprises the amino acid sequence from position 13 to position 292 of the amino acid sequence of pig uricase. In another embodiment, the uricase molecule comprising threonine, threonine, lysine, and serine, at positions 7, 46, 291, and 301 comprises the amino acid sequence from position 8 to position 287 of the amino acid sequence of Pig-KS-ΔN (Pig-TTKS-ΔN) (SEQ ID NO:7). In another embodiment, the uricase molecule comprising threonine, threonine, lysine, and serine, at positions 7, 46, 291, and 301 comprises the amino acid sequence from position 8 to position 287 of the amino acid sequence of Pig-KS-ΔN and the N-terminal methionine has been removed (Pig-TTKS-ΔN) (SEQ ID NO:8).

In an embodiment, the uricase molecule comprises the sequence of SEQ ID NO. 8 or a sequence 90%, 91%, 92%, 93%, 94%, 95%, 96%, 97%, 98%, 99%, or 100% identical, e.g., 95-98% identical thereto.

In embodiments, the uricase molecule comprises the amino acid sequence of des-(1-6)-[7-threonine, 46-threonine, 291-lysine, 301-serine]pig-baboon uricase.

The truncated mammalian uricase molecules disclosed herein can further comprise a methionine at the amino terminus. The penultimate amino acid can be one that allows removal of the N-terminal methionine by bacterial methionine aminopeptidase (MAP). Amino acids allowing the most complete removal of the N-terminal methionine are alanine, glycine, proline, serine, and threonine. In one embodiment, the uricase molecule comprises two amino terminal amino acids, wherein the two amino terminal amino acids are a methionine followed by an amino acid selected from the group consisting of alanine, glycine, proline, serine, and threonine. In another embodiment, the amino acid is threonine.

In an embodiment, the uricase molecule is a mammalian uricase molecule. Uricase genes and proteins have been identified in several mammalian species, for example, pig, baboon, rat, rabbit, mouse, and rhesus monkey. The sequences of various uricase proteins are described herein by reference to their public data base accession numbers, as follows: gi|50403728|sp|P25689; gi|20513634|dbj|BAB91555.1; gi|176610|gb|AAA35395.1; gi|20513654|dbj|BAB91557.1; gi|47523606|ref|NP_999435.1; gi|6678509|ret]NP_033500.1; gi|57463|emb|CAA31490.1; gi|20127395|rer]NP_446220.1; gi|137107|sp|P11645; gi|51458661|refjXP_497688.1; gi|207619|gb|AAA42318.1; gi|26340770|dbj|BAC34047.1; and gi|57459|emb|CAA30378.1. Each of these sequences and their annotations in the public databases accessible through the National Center for Biotechnology Information (NCBI) is incorporated by reference in its entirety.

In an embodiment, the mammalian uricase molecule comprises the sequence of porcine, bovine, ovine or baboon liver uricase.

In one embodiment, the uricase molecule can be baboon liver uricase in which tyrosine 97 has been replaced by histidine, whereby the specific activity of the uricase molecule can be increased by at least about 60%.

In an embodiment, the uricase molecule is a chimeric uricase of two or more mammalian uricases.

In an embodiment, the mammalian uricases of the chimeric uricase are selected from porcine, bovine, ovine, or baboon liver uricase.

In an embodiment, the uricase molecule comprises the sequence of SEQ ID NO. 8 or a sequence 90%, 91%, 92%, 93%, 94%, 95%, 96%, 97%, 98%, 99%, or 100% identical, e.g., 95-98% identical thereto.

In embodiments, the uricase molecule comprises the amino acid sequence of des-(1-6)-[7-threonine, 46-threonine, 291-lysine, 301-serine]pig-baboon uricase.

In embodiments, the uricase molecule is 80-200 kDa. In embodiments, the uricase molecule is 100-160 kDa. In embodiments, the uricase molecule is 110-150 kDa. In embodiments, the uricase molecule is 120-180 kDa. In embodiments, the uricase molecule is 130-140 kDa, e.g., about 135-137 kDa (e.g., about 136.8 kDa).

In one embodiment, the uricase molecule is isolated. In another embodiment, the uricase molecule is purified. In a further embodiment, the uricase molecule is isolated and purified.

The uricase molecule can be isolated and/or purified by any method known to those of skill in the art. Expressed polypeptides can generally be isolated in substantially pure form. In some embodiments, the polypeptides are isolated to a purity of at least 80% by weight. In further embodiments, the polypeptides are isolated to a purity of at least 95% by weight. In additional embodiments, the polypeptides are isolated to a purity of at least 99% by weight. In general, such purification can be achieved using, for example, the standard techniques of ammonium sulfate fractionation, SDS-PAGE electrophoresis, and affinity chromatography. The uricase molecule can be isolated using a cationic surfactant, for example, cetyl pyridinium chloride (CPC) according to the method described in United States patent publication US 2009/0317889, entitled Purification of Proteins With Cationic Surfactant, incorporated herein by reference in its entirety.

Methods for expressing the uricase molecule in a host cell are known in the art. For example, a host cell can be treated so as to cause the expression of the uricase molecule. Transfection of cells with a vector can be accomplished using DNA precipitated with calcium ions, though a variety of other methods can be used (e.g. electroporation). A vector expressing the uricase molecule can be under the control of an osmotic pressure sensitive promoter. A promoter is a region of DNA to which RNA polymerase binds before initiating the transcription of DNA into RNA. An osmotic pressure sensitive promoter initiates transcription as a result of increased osmotic pressure as sensed by the cell. In an embodiment, the promoter is a modified osmB promoter.

### Uricase Conjugates

In certain embodiments, the uricase molecule is in conjugate form. The uricase molecule disclosed herein can be conjugated to one or more polymers, e.g., water-soluble polymers (e.g., one or more PEG moieties).

Disclosed herein are conjugates, e.g., uricase conjugates, comprising uricase molecules. These conjugates are substantially non-immunogenic can and retain at least 75%, 85%, or 95% or more of the uricolytic activity of the unmodified enzyme. In embodiments, the polymer comprises one or more of a polyether, polyester, polyamino acid, a polycarbonate, a polyacetal, a polyketal, a polysaccharide, or a combination thereof. In embodiments, the polymer is polypropylene glycol. In embodiments, the polymer is polyethylene glycol. Water-soluble polymers suitable for use in the methods disclosed herein include linear and branched poly(ethylene glycols) or poly(ethylene oxides), all commonly known as PEGs. Examples of branched PEG are the subject of U.S. Pat. No. 5,643,575. One example of linear PEG is monomethoxyPEG, of the general structure CH3O—(CH2CH2O)nH, where n varies from about 100 to about 2,300.

In embodiments, each polymer molecule, e.g., polyethylene glycol molecule, has a molecular weight between about 1 kDa and 100 kDa. In embodiments, each polymer molecule, e.g., polyethylene glycol molecule, has a molecular weight between about 5 kDa and 50 kDa. In embodiments, each polymer molecule, e.g., polyethylene glycol molecule, has a molecular weight between about 5 kDa and 20 kDa. In embodiments, each polymer molecule, e.g., polyethylene glycol molecule, has a molecular weight of about 20 kDa. In embodiments, each polymer molecule, e.g., polyethylene glycol molecule, has a molecular weight of about 10 kDa. In embodiments, each polymer molecule, e.g., polyethylene glycol molecule, has a molecular weight of about 10 kDa.

In embodiments, the conjugate has a polymer weight of 300-800 kDa. In embodiments, the conjugate has a polymer weight of 400-700 kDa. In embodiments, the conjugate has a polymer weight of about 500-600 kDa, e.g., about 540-560 kDa.

In embodiments, the conjugate comprises 1 to 40 polymer molecules, e.g., polyethylene glycol molecules, per monomer of the uricase molecule. In embodiments, the conjugate comprises 5 to 20 polymer molecules, e.g., polyethylene glycol molecules, per monomer of the uricase molecule. In embodiments, the conjugate comprises 5 to 12 polymer molecules, e.g., polyethylene glycol molecules, per monomer of the uricase molecule. In embodiments, the conjugate comprises about 10 polymer molecules, e.g., polyethylene glycol molecules, per monomer of the uricase molecule.

Uricase molecules can be conjugated via a biologically stable, nontoxic, covalent linkage to a relatively small number of strands of PEG. Such linkages can include urethane (carbamate) linkages, secondary amine linkages, and amide linkages.

Urethane linkages to uricase can be formed by incubating uricase in the presence of the succinimidyl carbonate (SC) or p-nitrophenyl carbonate (NPC) derivative of PEG. SC-PEG can be synthesized using the procedure described in U.S. Pat. No. 5,612,460, which is hereby incorporated by reference. NPC-PEG can be synthesized by reacting PEG with p-nitrophenyl chloroformate according to methods described in Veronese, F M, et al., (1985) Appl Biochem Biotechnol 11:141-152, and in U.S. Pat. No. 5,286,637, which is hereby incorporated by reference. The methods described in U.S. Pat. No. 5,286,637 are adapted to PEGs of higher molecular weight by adjusting the concentrations of the reactants to maintain similar stoichiometry. An alternative method of synthesis of NPC-PEG is described by Buttner, W, et al., East German Patent Specification DD 279 486 A1. In embodiments, the uricase molecule of the conjugate is derivatized using monomethoxypoly(ethylene glycol)-p-nitrophenyl carbonate (mPEG-NPC).

Amide linkages to uricase can be obtained using an N-hydroxysuccinimide ester of a carboxylic acid derivative of PEG (Shearwater Polymers). Secondary amine linkages can be formed using 2,2,2-trifluoroethanesulfonyl PEG (tresyl PEG; Shearwater Polymers) or by reductive alkylation using PEG aldehyde (Shearvater Polymers) and sodium cyanoborohydride.

As described in U.S. Pat. No. 6,783,965, in conjugates containing PEG with a molecular weight of 10 kDa, the maximum number of strands of PEG that were coupled per subunit, while retaining at least 75% of the uricolytic activity of the unmodified enzyme, was about 12 strands for mammalian uricases (e.g. PKS uricase, a mutein of porcine uricase; see assay conditions in Example 5). The latter extent of PEGylation corresponds to about 40% of the total amino groups. There are several factors that can affect the choice of the optimal molecular weight and number of strands of PEG for coupling to a given form of uricase. In general, the reduction or elimination of immunogenicity without substantial loss of uricolytic activity can require the coupling of relatively more strands of PEG of lower molecular weight, compared to relatively fewer strands of PEG of higher molecular weight. Likewise, each different form of uricase can have a different optimum with respect to both the size and number of strands. The optimal number of strands of PEG and PEG molecular weight can be readily determined using art known methods.

In another embodiment, substantially all large aggregates of the uricase conjugate have been removed. Methods for removal include ion-exchange chromatography or size-exclusion chromatography at a pH between about 9 and 10.5, e.g., 10.2, prior to conjugation of the resulting substantially aggregate-free preparation of uricase to PEG. The molecular weight of the uricase in each fraction from the preparative column can be monitored by any size-dependent analytical technique, including, for example, HPLC, conventional size-exclusion chromatography, centrifugation, light scattering, capillary electrophoresis or gel electrophoresis in a non-denaturing buffer. For aggregate-free uricase isolated using size-exclusion chromatography, fractions containing only the 140-kDa and 280-kDa forms of the enzyme can be pooled and used for conjugation to PEG. For tetrameric plus octameric uricase isolated using ion-exchange chromatography, fractions from the ion-exchange column can be analyzed with respect to size to determine which fractions contain substantial amounts of the tetrameric and octameric forms without the large aggregates detected by light scattering. In the purified product, the undesirable large aggregates can thus constitute as little as about 1%, or less, of the total uricase.

In some embodiments, the uricase conjugate is pegloticase (KRYSTEXXA®). Pegloticase is a recombinant mammalian uricase conjugated to polyethylene glycol approved for the treatment of chronic gout in adult patients refractory to conventional therapy.¹ The recommended dose and regimen of pegloticase for adult patients is 8 mg (uricase protein) given as an intravenous infusion every two weeks.¹ Pre-infusion prophylaxis (antihistamines, corticosteroids) is indicated to minimize the risk of infusion reactions when administering pegloticase.¹ Hydrocortisone (200 mg IV) was administered during the randomized controlled trials, and has typically been used as a component of pre-infusion prophylaxis for pegloticase.² Methylprednisolone has been successfully used for pre-infusion prophylaxis for a number of biologic proteins including infliximab.³ Methylprednisolone is now being employed by some providers as an alternative pre-infusion prophylaxis corticosteroid for pegloticase. Methylprednisolone as prophylaxis for pegloticase infusion has not been systematically evaluated. Other types of corticosteroids that may be used as described herein may include, but are not limited to, predisone, hydrocortisone, methylprednisolone, Depo-Medrol, and/or Solu-Medrol.

### Uses of Combination Therapies

### Treatment

Elevated serum urate can be a biochemical marker of gout. Persistently elevated plasma uric acid (PUA) or serum uric acid (SUA) levels can result deposition of uric acid in joints and soft tissues. As the total body burden of uric acid increases, signs and symptoms of gout result, including arthritis, characterized by recurrent painful gout flares, development of tophi and joint deformities with resultant chronic pain/inflammation and consequent loss of physical function.

As used herein, and unless otherwise specified, the terms “treat,” “treating” and “treatment” refer to both therapeutic treatment and prophylactic or preventative measures, wherein the object is to prevent or slow down (lessen) an undesired physiological change or disorder, such as the development or worsening of a condition or disorder, e.g., hypeuricemia (e.g., gout). Beneficial or desired clinical results include, but are not limited to, alleviation of symptoms, diminishment of extent of disease, stabilized (i.e., not worsening) state of disease, delay or slowing of disease progression, amelioration or palliation of the disease state, whether detectable or undetectable. Those in need of treatment include those already with the condition or disorder, as well as those prone to have the condition or disorder or those in which the condition or disorder is to be prevented.

For example, in the case of treating gout, in some embodiments, therapeutic treatment can refer to reducing the uric acid level after administration in accordance with the methods or administration with the pharmaceutical compositions described herein. For example, the uric acid level is reduced by at least about 10%, at least about 15%, at least about 20%, at least about 30%, at least about 40%, or at least about 50%, after treatment. In another embodiment, the uric acid level is reduced by more than 50%, e.g., at least about 60%, or at least about 70%, after treatment. In one embodiment, the uric acid level is reduced by at least about 80%, at least about 90% or greater, as compared to a control (e.g. in the absence of the pharmaceutical composition described herein).

In another embodiment, the therapeutic treatment refers to alleviation of at least one symptom associated with gout. Measurable lessening includes any statistically significant decline in a measurable marker or symptom, such as serum/plasma uric acid levels in a blood sample, after treatment. In one embodiment, at least one marker or symptom is alleviated by at least about 10%, at least about 15%, at least about 20%, at least about 30%, at least about 40%, or at least about 50%. In another embodiment, at least one marker or symptom is alleviated by more than 50%, e.g., at least about 60%, or at least about 70%. In one embodiment, at least one marker or symptom is alleviated by at least about 80%, at least about 90% or greater, as compared to a control (e.g. in the absence of the pharmaceutical composition described herein).

As used herein, unless otherwise specified, the terms “prevent,” “preventing” and “prevention” refers to an action that occurs before the subject begins to have a detectably symptom or suffer from the condition, or relapse of the condition. In one embodiment, the condition is gout. Prevention need not result in a complete prevention of the condition; partial prevention or reduction of the condition or a symptom of the condition, or reduction of the risk of developing the condition, is encompassed by this term.

As used herein, and unless otherwise specified, a “therapeutically effective amount” of a compound, e.g., a uricase molecule in combination, is an amount sufficient to provide a therapeutic benefit in the treatment of the disorder (e.g., gout, or one or more symptoms associated with gout), or to delay or minimize one or more symptoms associated with the disorder (e.g., gout, or one or more symptoms associated with gout). A therapeutically effective amount of a compound means an amount of therapeutic agent, alone or in combination with other therapeutic agents, which provides a benefit in the treatment or management of the disorder. The term “therapeutically effective amount” can encompass an amount that improves overall therapy, reduces or avoids one or more symptoms or causes of the disorder (e.g., gout, or one or more symptoms associated with gout), or enhances the therapeutic efficacy of another therapeutic agent. In some embodiments, a therapeutically effective amount is an amount that achieves a reduction in uric acid levels. In some embodiments, a therapeutically effective amount is an amount that achieves uric acid levels less or about 6 mg/dl.

As used herein, and unless otherwise specified, a “prophylactically effective amount” of a compound is an amount sufficient to prevent a disorder (e.g., gout, or one or more symptoms associated with gout). A prophylactically effective amount of a compound means an amount of the compound, alone or in combination with other therapeutic agents, which provides a prophylactic benefit in the prevention of the disorder. The term “prophylactically effective amount” can encompass an amount that improves overall prophylaxis or enhances the prophylactic efficacy of another prophylactic agent.

As described herein, “gout” is a chronic disorder of urate metabolism resulting in deposition of monosodium urate crystals in the joints and soft tissues, with accompanying inflammation and eventually, in some patients, destructive, chronic arthropathy. Gout is a prevalent form of arthritis in men and is increasing in incidence and prevalence among older persons of both genders. Treatments for gout includes oral ULA (urate lowering agents), e.g., xanthine oxidase inhibitors, including allopurinol and febuxostat, and uricase. In some embodiments, patients who are unresponsive to oral ULA are treated with uricase.

“Refractory gout” refers to gout in a subject, e.g., a patient, who is unresponsive or poorly responsive to one or more oral urate-lowering agents (ULA), for example, xanthanine oxidase inhibitors, or have experienced or are at an increased risk of experiencing an adverse event therefrom. Approximately, 2% of patients treated with allopurinol develop allergic reactions and a severe hypersensitivity syndrome occurs in about 0.4% of the patients (Arellano et al., Ann Pharmacother 1993; 27:337-43). Patients with medical contraindications to xanthine oxidase inhibitors due to allergy/hypersensitivity, or who have failed to normalize SUA at maximum medically appropriate doses of these medications, can go on to develop chronic gout.

“Chronic gout refractory to Conventional Therapy (GRT)” is an uncommon, but severe outcome of progressive gout. Chronic gout results from demonstrated intolerance of or refractoriness to available therapy to prevent urate crystal deposition by reducing and maintaining serum urate levels in a subsaturating range.

Subjects who have repeated attacks of gout or persistent hyperuricemia for many years can develop tophaceous gout. Tophaceous gout describes the accumulation of large numbers of urate crystals in masses called tophi. Subjects with this form of gout can develop tophi in joints, bursae (the fluid-filled sacs that cushion and protect tissues), bones, and cartilage, or under the skin. Tophi may cause erosion of the bone and eventually joint damage and deformity (called gouty arthropathy).

In some embodiments, the uricase molecule used in the methods and dosage regimens disclosed herein is pegylated uricase. Pegylated uricase is a monomethoxypoly (ethylene glycol) (PEG) modified recombinant mammalian uricase (urate oxidase) which reduces levels of UA in the serum (or plasma) by catalyzing its conversion to allantoin, a water-soluble metabolite more readily excreted in the urine than uric acid. Pegylated uricase provides a therapeutic mechanism to reduce SUA in patients with gout. Pegylated uricase provides medical benefits in patients who respond by lowering SUA and by reducing tophus burden.

Described herein are tolerizing and therapeutic dosage regimens of uricase conjugates, e.g., pegylated uricase. Among other things, the combination of these dosing regimens provide for improved reduction of uric acid levels, improved treatment of gout, reduced antibody response, and prevention (e.g., delay of) infusion reactions relative to conventional dosage schemes.

### Infusion Reactions

In the case of PEGylated uricase therapy, it has been shown that infusion reactions (IRs) are associated with an increase in anti-PEGylated uricase antibody titer, loss of responsiveness to PEGylated uricase therapy, or both.

As used herein, the term “infusion reaction” is an undesired and/or unintended effect, e.g., an adverse event, of a therapy, e.g., a uricase therapy, occurring during or after the therapy. In some embodiments, the infusion reaction occurs during a uricase therapy. In other embodiments, the infusion reaction occurs after a uricase therapy. In further embodiments, the infusion reaction occurs within 2 hours after the uricase therapy.

In some embodiments, the uricase therapy comprises infusion, e.g., intravenous infusion, of uricase. An infusion reaction can be any adverse event. In some embodiments, an infusion reaction is a gout flare. In some embodiments, an infusion reaction comprises urticaria, dyspnea, chest discomfort, chest pain, erythema, or pruritus. In some embodiments, an infusion reaction is allergic. In some embodiments, an infusion reaction is non-allergic.

In some embodiments, an infusion reaction comprises anaphylaxis. Symptoms of anaphylaxis include generalized hives; pruritus or flushing; swollen lips, tongue, or uvula; Respiratory compromise (e.g., dyspnea, wheeze-bronchospasm, stridor, reduced peak expiratory flow, hypoxemia); and reduced blood pressure.

In some embodiments, an infusion reaction comprises an immune complex-related disorder. Immune complex-related disorders can be caused by the deposition of immune complexes in small blood vessels in the body. Immune complex-related disorders include glomerulonephritis, skin vasculitis, or arthritis.

### Combination Therapies

Disclosed herein are methods for treating gout, or reducing an infusion reaction, in response to a uricase therapy. In some embodiments, the methods disclosed herein include administration of a prednisone molecule and a uricase molecule in combination. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the uricase molecule, e.g., uricase conjugate, is administered at a dose of about 4 mg, about 5 mg, about 6 mg, about 7 mg, about 8 mg, or about 12 mg, e.g., about 8 mg.

In some embodiments, the prednisone molecule, e.g., the methylprednisolone, is administered in an amount ranging from about 0.1 mg/kg to about 5 mg/kg, about 0.2 mg/kg to about 4 mg/kg, about 0.3 mg/kg to about 3 mg/kg, about 0.4 mg/kg to about 2 mg/kg, or about 0.5 mg/kg to about 1.5 mg/kg, or typically about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, or about 1.5 mg/kg. In other embodiments, the methylprednisolone is administered as a flat dose, e.g., about 1 mg to about 500 mg, about 10 mg to about 400 mg, about 20 mg to about 300 mg, about 30 mg to about 200 mg, or about 50 mg to about 150 mg, or typically about 50 mg, about 60 mg, about 70 mg, about 80 mg, about 90 mg, about 100 mg, about 110 mg, about 120 mg, about 130 mg, about 140 mg, or about 150 mg.

In some embodiments, the prednisone molecule, e.g., the methylprednisolone, is administered prior to the uricase molecule. In some embodiments, the prednisone is administered about 10 minutes or more, about 20 minutes or more, about 30 minutes or more, about 40 minutes or more, about 50 minutes or more, about 1 hour or more, about 2 hours or more, or about 3 hours or more before administration of the uricase molecule. In other embodiments, the methylprednisone is administered about 10 minutes, about 20 minutes, about 30 minutes, about 40 minutes, about 50 minutes, about 1 hour, about 2 hours, or about 3 hours before administration of the uricase molecule. In other embodiments, the administration of the prednisone and the uricase molecule partially overlap.

In one embodiment, the prednisone molecule is administered intravenously (IV), intramuscularly (IM), by infusion (IV infusion), orally, rectally, or topically.

In some embodiments, the treatment cycle is repeated once every 11 days, 12 days, 13 days, 14 days, 15 days, 16 days, 17 days, or 18 days, e.g., every 14 days, e.g., every other week. In one embodiment, dosage regimen is repeated once every other week.

In some embodiments, the dosage regimen comprises one or more first treatment cycles of prednisone and a uricase molecule, and one or more second treatment cycles of a uricase molecule without prednisone.

In some embodiments, dosage regimen includes 7, 10, 12, 24, 48, or more treatment cycles.

### Subjects

The methods described herein are useful for treating subjects, e.g., human subjects. In some embodiments, the subject is a patient, e.g., a human patient. In some embodiments, the subject has gout, e.g., is a patient with gout. In some embodiments, the subject is diagnosed with gout. In some embodiments, the subject treated shows hyperuricemia, e.g., the subject has gout. In certain embodiments, the subject is refractory, or has reduced responsiveness, to a urate lowering therapy, e.g., oral ULA. In one embodiment, the subject has, or is identified as having, a lower efficacy to a uricase therapy (e.g., shows increased incidence of anti-uricase antibodies (e.g., anti-pegloticase antibodies) and/or increased incidence of an infusion reaction in response to the uricase therapy). In some embodiments, the subject is overweight or obese, e.g., has a baseline body weight of >100 kg.

A diagnosis of gout can be based on inflamed joints as determined by MRI, X-ray, or ultrasound, the presence of crystals in the synovial fluid analysis, elevated serum uric acid as determined by blood or urine test, and/or tophi. In some embodiments, the subject is likely to have gout. In some embodiments, gout is chronic. In some embodiments, gout is tophaceous gout. In some embodiments, gout is acute. In some embodiments, gout is severe. In some embodiments, gout is moderate.

In some embodiments, the subject has refractory gout, e.g., has failed to normalize serum uric acid, and/or has signs and symptoms inadequately controlled with ULA, e.g., xanthanine oxidase inhibitors. In some embodiments, the subject was previously treated with oral ULA, e.g., xanthanine oxidase inhibitors, allopurinol, or febuxostat.

The serum urate saturation point can be approximately 6.8 mg/dL. Without wishing to be bound by theory, it is thought that the risk for the development of gout symptoms increases steadily at concentrations higher than 6 mg/dL. In some embodiments, the subject is hyperuricemic, e.g., has a serum uric acid level ≥4 mg/dl, ≥4.1 mg/dl, ≥4.2 mg/dl, ≥4.3 mg/dl, ≥4.4 mg/dl, ≥4.5 mg/dl, ≥4.5 mg/dl, ≥4.6 mg/dl, ≥4.7 mg/dl, ≥4.8 mg/dl, ≥4.9 mg/dl, ≥5.0 mg/dl, ≥5.1 mg/dl, ≥5.2 mg/dl, ≥5.3 mg/dl, ≥5.4 mg/dl, ≥5.5 mg/dl, ≥5.6 mg/dl, ≥5.7 mg/dl, ≥5.8 mg/dl, ≥5.9 mg/dl, ≥6.0 mg/dl, ≥6.1 mg/dl, ≥6.2 mg/dl, ≥6.3 mg/dl, ≥6.4 mg/dl, ≥6.5 mg/dl, ≥6.6 mg/dl, ≥6.7 mg/dl, ≥6.8 mg/dl, e.g., has a serum uric acid level ≥6 mg/dl.

One category of patients who can benefit from the claimed methods are overweight or obese patients. Without wishing to be bound by theory, in such patients, the dosage of the uricase conjugate expressed as mg/kg can be reduced relative to a normal weight patient and this can reduce the efficacy of the treatment. A therapeutic dosage regimen with increased dosage or increased frequency of dosages at the beginning of a treatment period can increase responsiveness to uricase therapy in these patients. As used herein, “treatment period” refers to the length or duration of treatment for a particular individual, i.e., the length of time a particular patient or individual is given a uricase molecule for lowering uric acid levels. Treatment period may refer to a period of weeks of administration of a uricase molecule as described herein, or may refer to the length of time of an individual administration (i.e., infusion) of a uricase molecule. Treatment period may vary depending on the individual as described herein. Treatment period also refers to the period of administration of a steroid, either prior to, concurrently with, or after administration of a uricase molecule in a patient. A treatment period may vary depending on the individual In some embodiments, the subject has a body weight greater than or equal to 80 kg, 90 kg, 100 kg, 110 kg, 120 kg, e.g., 100 kg. In some embodiments, the subject has been identified as having a body weight greater than or equal to 80 kg, 90 kg, 100 kg, 110 kg, 120 kg, e.g., 100 kg.

Assessment of overweight and obesity can be determined by the classification of body mass index (BMI) as defined by “Clinical Guidelines on the Identification, Evaluation, and Treatment of Overweight and Obesity in Adults” from the National Institutes of Health. Body mass index is obtained by dividing a subject's weight, e.g., in kilograms (kg) by the square of the subject's height, e.g., in meters (m). Subjects with a BMI 18.5 to 24.9 are typically classified as normal weight, while subjects with a BMI 25.0 to 29.9 are classified as overweight. Subjects with a BMI 30.0 or greater are classified as obese, and can be subdivided into three classes: Class I (BMI=30.0 to 34.9; Class II (BMI=35.0 to 39.9); and Class III (BMI is greater or equal to 40).

In one embodiment, the subject is overweight, e.g., the subject has a BMI of greater than or equal to 25.0 but less than or equal to 29.9.

In another embodiment, the subject is, or is identified as being, obese, e.g., the subject has a BMI of greater than or equal to 30, e.g., greater than 30, greater than 35, greater than 40, greater than 45, or greater than 50.

In some embodiments, the subject is 55 years old or older, 60 years old or older, 65 years old or older, or 70 years old or older, e.g., 65 years old or older. In some embodiments, the subject is younger than 70, 65, 60, or 55 years old, e.g., younger than 65 years old.

### Monitoring of Subjects

Suitable indicators for assessing effectiveness of a given dosage regimen are known to those of skill in the art. Examples of such indicators include normalization or lowering of serum uric acid (SUA) or plasma uric acid levels (PUA) and lowering or maintenance of the uric acid level. It is known that persistently elevated plasma uric acid (PUA) or serum uric acid (SUA) levels result in deposition of uric acid in joints and soft tissues. As the total body burden of uric acid increases, signs and symptoms of gout result, including arthritis, characterized by recurrent painful gout flares, development of tophi and joint deformities with resultant chronic pain/inflammation and consequent loss of physical function.

In embodiments, the uric acid level, e.g., the serum or plasma uric acid level, can be lowered to 6.8 mg/dL or less, 6.7 mg/dL or less, 6.6 mg/dL or less, 6.5 mg/dL or less, 6.4 mg/dL or less, 6.3 mg/dL or less, 6.2 mg/dL or less, 6.1 mg/dL or less, 6.0 mg/dL or less, 5.9 mg/dL or less, 5.8 mg/dL or less, 5.7 mg/dL or less, 5.6 mg/dL or less, 5.5 mg/dL or less, 5.4 mg/dL or less, 5.3 mg/dL or less, 5.2 mg/dL or less, 5.1 mg/dL or less, 5.0 mg/dL or less, 4.9 mg/dL or less, 4.8 mg/dL or less, 4.7 mg/dL or less, 4.6 mg/dL or less, 4.5 mg/dL or less, 4.4 mg/dL or less, 4.3 mg/dL or less, 4.2 mg/dL or less, 4.1 mg/dL or less, or 4.0 mg/dL or less. In embodiments, the uric acid level, e.g., the serum or plasma uric acid level, can be lowered to 6.8 mg/dL or less. In embodiments, the uric acid level, e.g., the serum or plasma uric acid level, can be lowered to 6 mg/dL or less. In embodiments, the uric acid level is measured before dose of the conjugate and the uric acid level can be lowered to 6 mg/dl or less for 3 or more consecutive doses. In embodiments, the uric acid level, e.g., the serum or the plasma uric acid level, can be lowered to 5.0 mg/dl or less. In embodiments, the subject being treated with the compositions disclosed herein has a SUA or PUA of 6 mg/ml or less for at least 70%, at least 80%, or at least 90% of the total treatment period. For example, for a 24-week treatment period, the subject has a SUA or PUA of 6 mg/ml or less for at least 80% of the 24 week treatment period, i.e., for at least a time equal to the amount of time in 134.4 days (24 weeks×7 days/week×0.8=134.4 days). In embodiments, the uric acid level, e.g., the serum or plasma uric acid level, can be reduced in the subject relative to a reference, e.g., relative to an untreated subject, or the subject prior to treatment or after a previous treatment.

In embodiments, the uric acid level can be measured by an immunosorbant assay, e.g., enzyme-linked immunosorbant assay (ELISA). In embodiments, a trough level of uric acid can be measured before administration of the uricase conjugate, e.g., 72, 48, 24, 12, 6, 4, or 2 hours before administration of the conjugate. In embodiments, a peak level of uric acid can be measured after administration of the conjugate, e.g., 0.5, 1, 2, 3, 4, or 5 hours after administration of the conjugate.

Another measure of effectiveness of a given dosage regimen is measuring the level of an antibody against the conjugate in the subject. Anti-conjugate antibodies, e.g., anti-PEGylated uricase antibodies, can be measured, for example, in the blood of a subject. Methods for measuring levels of antibodies are well known in the art and include, for example, enzyme-linked immunosorbant assays (ELISAs). In embodiments, the level of the antibody against the conjugate can be measured before administration of the conjugate, e.g., 72, 48, 24, 12, 6, 4, or 2 hours before administration of the conjugate. In embodiments, the level of the antibody against the conjugate can be measured after administration of the conjugate, e.g., 0.5, 1, 2, 3, 4, or 5 hours after administration of the conjugate.

Another measure of effectiveness of a given dosage regimen is measuring is the Health Assessment Questionnaire Disability Index (HAQ-DI) in the subject. The HAQ-DI is designed to assess level of functional ability in 8 categories (Bruce and Fries, 2003). It consists of 20 questions representing a comprehensive set of functional activities (dressing and grooming, arising, eating, walking, hygiene, reach, grip, and activities). Responses are scored on a scale from 0 (no disability) to 3 (completely disabled). The HAQ also includes self-assessments of severity of pain and overall functional ability. In embodiments, the HAQ-DI of the subject can be reduced relative to a reference. In some embodiments, the reference is the subject at an earlier time point. In some embodiments, the earlier time point is prior to treatment. In some embodiments, the earlier time point is immediately prior to treatment. In some embodiments, the earlier time point is earlier in treatment. In some embodiments, the earlier time point is after a previous treatment. In embodiments, the HAQ-DI of the subject can be reduced by 1 point or more in 1, 2, 3 or more categories.

Another measure of effectiveness of a given dosage regimen is the level of C-reactive protein in the subject. C-reactive protein (CRP) is an annular (ring-shaped), pentameric protein found in blood plasma, whose levels rise in response to inflammation, including inflammatory conditions such as gout. Methods for measuring levels of proteins, e.g., CRP, are well known in the art. In embodiments, the level of C-reactive protein can be reduced relative to a reference. In some embodiments, the reference is a subject is an untreated subject. In some embodiments, the reference is a subject being treated with uricase. In some embodiments, the subject is receiving only the therapeutic dosage regimen, e.g., 8 mg of polyethylene glycol-uricase conjugate every two weeks.

In embodiments, the level of C-reactive protein can be reduced relative to the level of C-reactive protein in the subject at an earlier time point. In some embodiments, the earlier time point is prior to administration of the conjugate. In some embodiments, the earlier time point is immediately prior to administration of the conjugate. In some embodiments, the level of C-reactive protein can be reduced by 10%, 20%, 30%, or more, relative to the levels of C-reactive protein in the subject prior to administration of the conjugate.

Another measure of effectiveness of a given dosage regimen is the frequency of gout flares. In embodiments, the subject has a decreased frequency of gout flares. In embodiments, the subject has a decreased frequency of gout flares relative to a reference. In some embodiments, the reference is a subject is an untreated subject. In some embodiments, the reference is a subject being treated with uricase. In some embodiments, the subject is receiving only the therapeutic dosage regimen, e.g., 8 mg of polyethylene glycol-uricase conjugate every two weeks.

In embodiments, the subject shows a decreased in the frequency of gout flares relative to the frequency of gout flares in the subject relative to an earlier time point. In some embodiments, the earlier time point is an earlier time during treatment with the conjugate. In some embodiments, the earlier time point is prior to treatment with the conjugate. In some embodiments, the earlier time point is immediately prior to treatment with the conjugate. In embodiments, the subject shows a 5%, 10%, 20%, 30%, 40%, 50%, 60%, 70%, 80%, or 90% reduction in the frequency of gout flares relative to the frequency of gout flares in the subject prior to administration of the conjugate.

### Pharmaceutical Compositions

Pharmaceutical formulations containing uricase molecules or uricase conjugates, e.g., pegylated uricase, can be prepared by conventional techniques, e.g., as described in Gennaro, AR (Ed.) (1990) Remington's Pharmaceutical Sciences, 18th Edition, Easton, Pa.: Mack Publishing Co. Suitable excipients for the preparation of injectable solutions include, for example, phosphate buffered saline, lactated Ringer's solution, water, polyols and glycerol. Pharmaceutical compositions for parenteral injection comprise pharmaceutically acceptable sterile aqueous or non-aqueous liquids, dispersions, suspensions, or emulsions as well as sterile powders for reconstitution into sterile injectable solutions or dispersions just prior to use. These formulations can contain additional components, such as, for example, preservatives, solubilizers, stabilizers, wetting agents, emulsifiers, buffers, antioxidants and diluents.

Uricase molecules or uricase conjugates can also be provided as controlled-release compositions for implantation into an individual to continually control elevated uric acid levels in body fluids. For example, polylactic acid, polyglycolic acid, regenerated collagen, poly-L-lysine, sodium alginate, gellan gum, chitosan, agarose, multilamellar liposomes and many other conventional depot formulations comprise bioerodible or biodegradable materials that can be formulated with biologically active compositions. These materials, when implanted or injected, gradually break down and release the active material to the surrounding tissue. For example, one method of encapsulating PEG-uricase comprises the method disclosed in U.S. Pat. No. 5,653,974, which is hereby incorporated by reference. In some embodiments, the conjugate is provided as a bioerodible, biodegradable or other depot formulation. In embodiments, infusion pumps and matrix entrapment systems are used for delivery of uricase molecules or uricase conjugates. Uricase molecules or uricase conjugates can also advantageously be enclosed in micelles or liposomes. Liposome encapsulation technology is well known in the art. See, e.g., Lasic, D, et al., (Eds.) (1995) Stealth Liposomes. Boca Raton, Fla.: CRC Press.

In some embodiments, the uricase conjugate is provided in a nanocarrier formulation. In some embodiments, the nanocarrier formulation comprise a lipid nanoparticle, a liposome, a polymeric nanoparticle, a dendrimer, or a surfactant based emulsion. In some embodiments, the polymeric nanoparticle comprises one or more of a polyether, polyester, polyamino acid, a polycarbonate, a polyacetal, a polyketal, a polysaccharide, or a combination thereof.

In some embodiments, the nanocarrier formulation comprises an immunosuppressant. In some embodiments, the immunosuppressant is an mTOR inhibitor, e.g., rapamycin or a rapamycin analogue.

The compositions disclosed herein can decrease the need for hemodialysis in patients at high risk of urate-induced renal failure, e.g., organ transplant recipients (see Venkataseshan, V S, et al., (1990) Nephron 56:317-321) and patients with some malignant diseases. In patients with large accumulations of crystalline urate (tophi), such pharmaceutical compositions can improve the quality of life more rapidly than currently available treatments.

In one embodiment, the composition is a solution of uricase. In another embodiment, the solution is sterile and suitable for injection. In one embodiment, such composition comprises uricase as a solution in phosphate buffered saline. In one embodiment, the composition is provided in a vial, optionally having a rubber injection stopper. In some embodiments, the composition comprises uricase in solution at a concentration of from 2 to 16 milligrams of uricase per milliliter of solution, from 4 to 12 milligrams per milliliter or from 6 to 10 milligrams per milliliter. In another embodiment, the composition comprises uricase at a concentration of 8 milligrams per milliliter. In embodiments, the mass of uricase is measured with respect to the protein mass.

The uricase can be administered in any appropriate way known to one of skill in the art, for example, intravenously, intramuscularly or subcutaneously. In some embodiments, when the administration is intravenous, 0.5 mg to 12 mg of uricase is administered. In further embodiments, when the administration is subcutaneous, 4 to 24 mg of uricase is administered. In one embodiment, the uricase is administered by intravenous infusion over a 30 to 240 minute period. In some embodiments, the infusion can be performed using 100 to 500 mL of saline solution. In some embodiments, the uricase is dissolved in 250 mL of saline solution for infusion. In some embodiments, saline solution comprises 0.45% or 0.9% sodium chloride. In some embodiments, the uricase administrations take place over a treatment period of 3 months, 6 months, 8 months or 12 months. In other embodiments, the treatment period is 12 weeks, 24 weeks, 36 weeks or 48 weeks. In one embodiment, the treatment period is for an extended period of time, e.g., 2 years or longer, for up to the life of subject being treated. In addition, multiple treatment periods can be utilized interspersed with times of no treatment, e.g., 6 months of treatment followed by 3 months without treatment, followed by 6 additional months of treatment, etc.

### EXAMPLES

### Example 1: Prophylaxis for Infusion Reactions to Pegloticase: An Analysis of Two Different Corticosteroid Pre-Infusion Regimens in US Community Rheumatology Practices

The effects on duration of therapy and infusion reactions in pegloticase administration were studied using methylprednisolone compared to hydrocortisone for pre-infusion prophylaxis. It was hypothesized that use of methylprednisolone compared to hydrocortisone will lead to fewer infusion reactions and longer duration of therapy.

### Design and Procedures

Retrospective chart abstraction was performed of patients treated with pegloticase between January 2013 and March 2016 and whose treatment course was complete. Charts of 92 qualifying patients were included in the data analysis. Nineteen geographically-dispersed US community-based rheumatology practices were represented in the analysis sample.

The variables collected included demographics, co-morbidities, duration of disease, sUA (prior to each infusion), pegloticase infusion dates and times, pre-infusion prophylaxis (including both steroids and antihistamines), and pre- and post-pegloticase gout therapies.

### Data Analysis

The number of infusions was treated as the response variable. Age, sUA prior to the last infusion and type of pre-infusion corticosteroid (hydrocortisone or methylprednisolone) were treated as predictor variables. Analyses were carried out by multiple logistic regression with significance p<0.01 as the accepted significance level.

### Results

Data from 96 patients were collected. Of these, the data from 92 patients met study inclusion criteria. The records of three patients were dropped from the analyses due to failure to meet study inclusion criteria, and one patient represented an extreme outlier. The demographic characteristics of the study sample (summarized in Table 1) were similar to the general population of patients with chronic refractory gout. The mean dose of hydrocortisone used conforms with the standard pre-infusion prophylaxis used during the randomized controlled clinical trials.

TABLE 1
Patient Demographics and Characteristics.

| | | |
|---|---|---|
| Age (years) | Mean | 61.5 |
| | Range | 38-77 |
| Sex, n (%) | Male | 74 (82%) |
| | Female | 18 (18%) |
| Race, n (%) | Caucasian | 73 (79%) |
| | Black | 11 (12%) |
| | Asian | 6 (7%) |
| | Other/NA | 1 (1%) |
| Disease Duration | Mean | 13.5 |
| (years) | Range | 4-30 |
| Number of Infusions (n) | Median Per Patient | 7 |
| | Total | 670 |

Corticosteroid

| Hydrocortisone | Number of patients (n) | 31 | |
|---|---|---|---|
| | Mean Dose | 198 | mg |
| | Modal Dose | 200 | mg |
| | Range | 150-200 | mg |
| Methylprednisolone | Number of patients (n) | 61 | |
| | Mean Dose | 77 | mg |
| | Modal Dose | 50 | mg |
| | Range | 40-120 | mg |

### Regression Analysis

Multiple logistic regression analysis was conducted using type of corticosteroid prophylaxis, patient age (<65 vs ≥65) and terminal sUA (sUA prior to final infusion) as predictor variables with duration of therapy (number of infusions) as the dependent variable. Use of methylprednisolone and terminal sUA were highly significant (p<0.001) predictors of therapy duration. Use of methylprednisolone entered the model first as a predictor of therapy duration. In a linear model containing both type of corticosteroid prophylaxis and pre-infusion sUA, pre-infusion sUA was a significant predictor of infusion reaction (p<0.001). There was no significant difference between sUA levels prior to initiation of pegloticase therapy for the two groups. In a linear model containing just type of corticosteroid, type of corticosteroid prophylaxis was a significant (p<0.001) predictor of infusion reaction.

### Number of Infusions

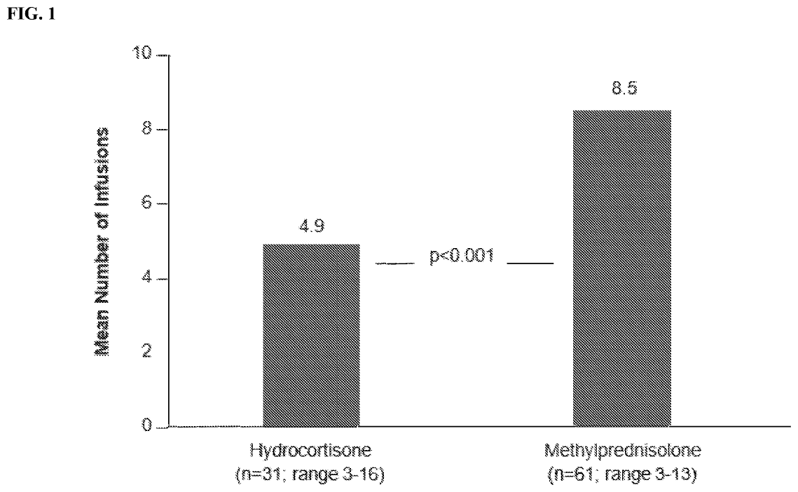

Mean number of pegloticase infusions for patients receiving methylprednisolone as prophylaxis was significantly (p<0.001) higher than for patients receiving hydrocortisone as prophylaxis (FIG. 1). The range of pegloticase infusions was 3-16 for patients receiving hydrocortisone and 3-13 for patients receiving methylprednisolone.

### Discontinuation of Therapy

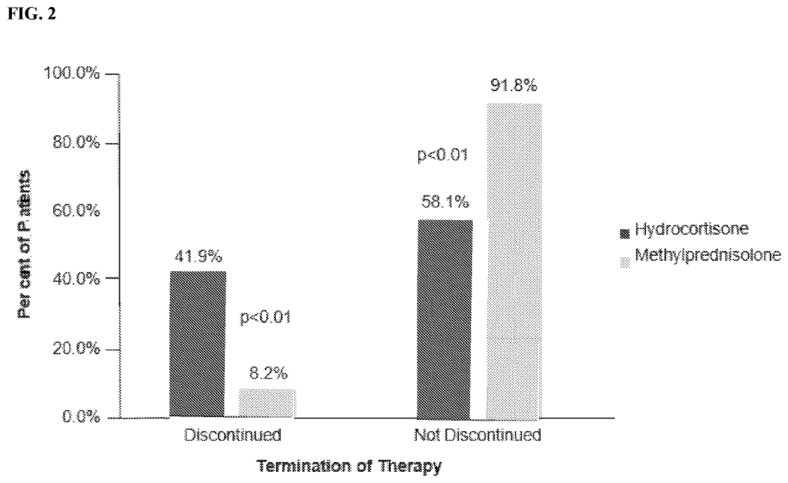

Discontinuations of therapy prior to planned treatment end occurred for a number of reasons, including patient choice and infusion reaction. The most frequent reason for premature treatment termination was the presence of an infusion reaction during an infusion. All infusion reactions were characterized as mild or moderate with symptoms similar to the pegloticase pivotal trials. When infusion reactions occurred, infusions were either managed by stopping the infusions or restarting at a slower rate. A significantly lower proportion of patients receiving methylprednisolone as prophylaxis terminated therapy early (8.2%) vs patients receiving hydrocortisone as prophylaxis (41.9%, p<0.01) (FIG. 2).

### Number of Infusions by Termination of Therapy

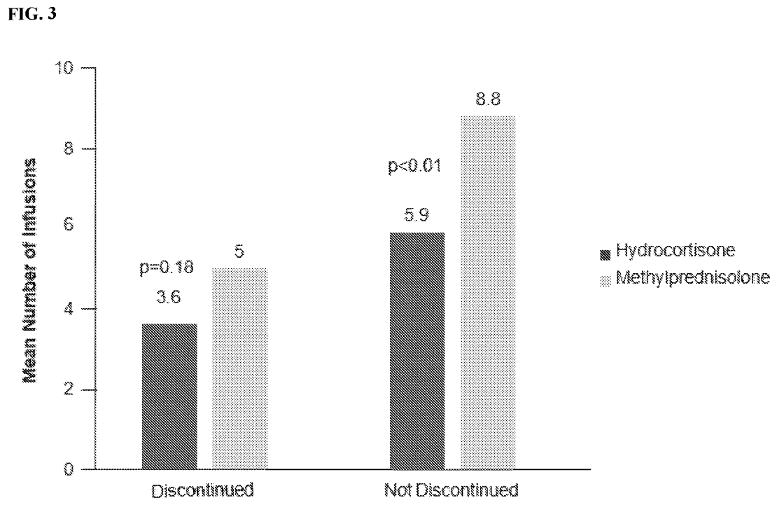

The mean number of infusions was greater for both types of prophylaxis if therapy was continued to the planned duration rather than being terminated by patient choice or infusion reaction (p<0.01). However, for both outcomes, the mean number of infusions was greater for patients receiving methylprednisolone for prophylaxis. The difference in the mean number of infusions for therapy discontinuation was not significant (p=0.18), most likely due to the small n for patients discontinuing on methylprednisolone. For therapy not discontinued, the difference in the mean number of infusions was significant (p<0.01) (FIG. 3).

### CONCLUSIONS

Patients in the study received a significantly greater number of infusions when methylprednisolone was used as the corticosteroid for pre-infusion prophylaxis. These results indicate that methylprednisolone for pre-infusion prophylaxis may allow for longer pegloticase therapy duration compared to hydrocortisone. This modality imposes a significantly higher glucocorticoid load that may suppress symptoms of infusion reaction and/or inhibit the formation of pegloticase drug antibodies. Methylprednisolone compared to hydrocortisone as a pre-infusion prophylaxis for pegloticase needs further study to determine both efficacy and long-term safety.

### EQUIVALENTS

Those skilled in the art will recognize, or be able to ascertain using no more than routine experimentation, many equivalents to the specific embodiments of the disclosure described herein.

1-81. (canceled)

82. A method of treating gout in a subject in need thereof, comprising repeatedly administering to the subject both pegloticase and methylprednisolone during a treatment period, wherein the methylprednisolone is administered as a pre-dose prophylaxis prior to each administration of pegloticase;
wherein 4 to 12 mg of pegloticase and 40 to 120 mg of methylprednisolone are administered once every 14 days during the treatment period; and
wherein no serum uric acid lowering therapeutic besides pegloticase is administered during the course of administering pegloticase.

83. The method of claim 82, wherein the subject was previously treated with one or more of an oral urate-lowering therapy, a xanthine oxidase inhibitor, allopurinol, and febuxostat.

84. The method of claim 82, wherein a second non-uricase urate-lowering therapy is administered after discontinuing the administration of pegloticase at the end of the treatment period

85. The method of claim 84, wherein the second non-uricase urate-lowering therapy is an oral urate-lowering agent (ULA) selected from a xanthine oxidase inhibitor, allopurinol, and febuxostat.

86. The method of claim 82, wherein the treatment period is 24 weeks.

87. The method of claim 82, wherein the treatment period is six months.

88. The method of claim 82, wherein the treatment period is twelve months.

89. The method of claim 82, wherein 12 doses of pegloticase are administered.

90. The method of claim 82, wherein 24 doses of pegloticase are administered.

91. The method of claim 82, wherein the serum uric acid levels are reduced to less than 6 mg/dL as a result of treatment with pegloticase.

92. The method of claim 82, wherein the pegloticase is administered by intravenous infusion.

93. The method of claim 82, wherein the methylprednisolone is administered by intravenous infusion.

94. The method of claim 82, wherein both the pegloticase and the methylprednisolone are administered by intravenous infusion.

95. The method of claim 82, wherein the methylprednisolone is administered immediately prior to the administration of the uricase molecule.
